(19) United States
(12) Patent Application Publication
(10) Pub. No.: US 2006/0069303 A1
(43) Pub. Date: Mar. 30, 2006

(54) Endoscopic apparatus with integrated hemostasis device

(76) Inventor: Lucien Alfred Couvillon JR., Concord, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/955,908

(22) Filed: Sep. 30, 2004

(51) Int. Cl. A61B 1/00; A61B 18/18

(52) U.S. Cl. 600/104; 600/106; 600/129; 606/46

(57) ABSTRACT

An endoscope or other medical device includes an elongated shaft having a flexible proximal section, an articulatable distal region, and distal tip. The endoscope or other device includes an associated hemostasis device for treating internal bleeding during a contemporaneous medial procedure. Embodiments of the present invention may incorporate mechanical, chemical, and/or electrical techniques for performing hemostasis.

### FIELD OF THE INVENTION

The present invention is generally directed to endoscopic apparatuses for use in medical procedures, and in particular, to endoscopic apparatuses having associated hemostasis devices.

### BACKGROUND OF THE INVENTION

It has become well established that there are major public health benefits from regular endoscopic examinations as an aid to the early detection of disease of internal structures such as the alimentary and excretory canals and airways, e.g., the colon, esophagus, lungs, uterus, bladder, bronchi, and other organ systems. A conventional imaging endoscope used for such procedures comprises a flexible tube with a fiber optic light guide that directs illuminating light from an external light source to the distal tip where it illuminates the region (i.e. tissue, occlusion object) to be examined. Frequently, additional optical components are incorporated to adjust the spread of the light exiting the fiber bundle and the distal tip. An objective lens and fiber optic imaging light guide communicating with a camera at the proximal end of the scope, or an imaging camera chip at the distal tip, produce an image that is displayed to the operator. In addition, most endoscopes include one or more working channels through which medical devices such as biopsy forceps, snares, fulguration probes, and other tools may be passed.

Once the endoscope is in position, tools inserted through or associated with the endoscope can be brought to the proper position in the tract or cavity of the body being examined, such as the GI tract. Various procedures can then be carried out, such as removing polyps, irrigation, suction, and removing other tissues. The various tools that are used together with the endoscope can be either inserted separately in the tract or cavity and placed in the proper position independently, or may travel in a working channel of the endoscope, so that once the endoscope is positioned at the desired location in the tract or cavity, the tools may be inserted in the endoscope and easily routed to the desired position.

One such tool that is frequently routed through the working channel of an endoscope is an RF electrode probe for performing hemostasis. Such a tool is utilized in such procedures as treating upper GI bleeding. Upper GI bleeding may be caused by esophageal varices or various upper GI ulcers. Generally described, gastroscopes, bronchoscopes or other upper GI endoscopes may be used to diagnose and locate bleeding vessels in patient passageways. Once located, a discrete hemostasis radio frequency (RF) probe, such as the Gold Probe commercially available from Boston Scientific, is routed through the working channel of the scope and activated to seal off the bleeder. While this method may be effective in treating internal bleeding, it is not without its deficiencies. For example, the aforementioned procedure is tedious and time consuming because of the need to introduce, position, energize, and withdraw the RF probe if other implements, e.g. biopsy forceps, are needed between the treatment of two bleeders.

### SUMMARY OF THE INVENTION

In accordance with aspects of the present invention, an endoscope is provided. The endoscope includes an elongated shaft body having a proximal end and a distal end, a distal tip section coupled to the distal end of the body, and a hemostasis device carried on the body and positioned proximal the distal tip section.

In accordance with another aspect of the present invention, an endoscope is provided. The endoscope includes an elongated, flexible body having a proximal end and a distal end, a distal tip coupled to the distal end of the body, and hemostasis means carried on the body and positioned proximal the distal tip.

In accordance with still another aspect of the present invention, a method of treatment using an endoscope is provided. The method includes routing an endoscope having an associated hemostasis device through a passageway to an internal wound site and performing hemostasis at the internal wound site with the hemostasis device.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is a partial perspective view of the distal region of one exemplary embodiment of an endoscope formed in accordance with aspects of the present invention;

FIG. 2 is a partial perspective view of the distal region of another exemplary embodiment of an endoscope formed in accordance with aspects of the present invention;

FIG. 3 is a partial perspective view of the distal region of still another exemplary embodiment of an endoscope formed in accordance with aspects of the present invention;

FIG. 4 is a partial perspective view of the distal region of yet another exemplary embodiment of an endoscope formed in accordance with aspects of the present invention;

FIG. 5 is a side partial cross-sectional view of the distal end of still yet another exemplary embodiment of an endoscope formed in accordance with aspects of the present invention;

FIGS. 6 and 7 illustrate partial side views of the distal region of exemplary embodiments of endoscopes configured for treating an existing blood clot in a patient's passageway in accordance with aspects of the present invention; and

FIGS. 8 and 9 illustrate partial perspective views of an alternative embodiment of an endoscope configured for treating an existing blood clot in a patient's passageway in accordance with aspects of the present invention.

### DETAILED DESCRIPTION OF THE PREFERRED EMBODIMENT

The present invention will now be described with reference to the drawings where like numerals correspond to like elements. Embodiments of the present invention are directed to devices of the type broadly applicable to numerous medical applications in which it is desirable to insert an imaging device, catheter or similar device into a body lumen or passageway. Specifically, embodiments of the present invention are directed to medical devices having hemostasis capabilities. Several embodiments of the present invention are directed to medical devices having hemostasis capabilities that incorporate endoscopic features, such as illumination and visualization capabilities, for endoscopically viewing anatomical structures within the body. As such, embodiments of the present invention can be used for a variety of different diagnostic and interventional procedures, including colonoscopy, upper endoscopy, bronchoscopy, thoracoscopy, laparoscopy and video endoscopy, etc., and are particularly well suited for negotiating tortuous passageways of the human body. Although exemplary embodiments of the present invention will be described hereinafter as endoscopes, it will be appreciated that aspects of the present invention have wide application, and may be incorporated into other medical devices, such as catheters, where hemostasis capabilities are desirable. Accordingly, the following descriptions and illustrations herein should be considered illustrative in nature, and thus, not limiting the scope of the present invention, as claimed.

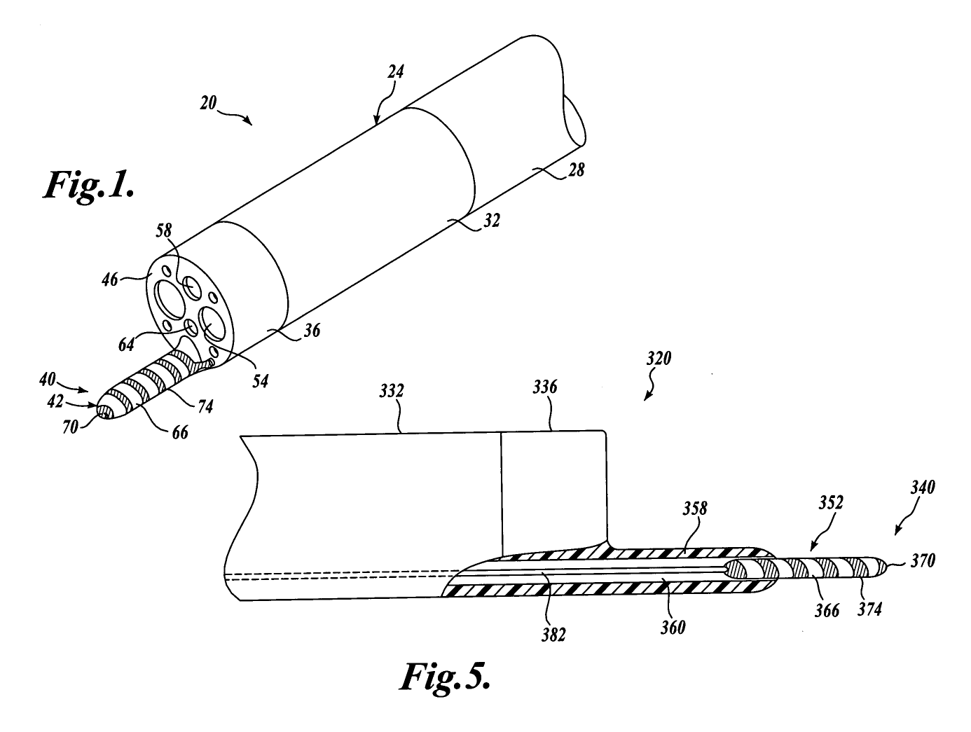

FIG. 1 illustrates a partial perspective of one embodiment of a medical device, and in particular, an endoscope 20 constructed in accordance with aspects of the present invention. The endoscope 20 includes an elongated tubular body 24, also known as an insertion tube, having a flexible proximal section 28, an optional articulatable distal region 32, and a distal tip 36. As will be described in more detail below, the endoscope 20 includes an associated hemostasis device 40, such as an electrode probe, clip device, suturing device, etc., for treating internal bleeding during or subsequent the medial procedure. As will be described in more detail below, embodiments of the present invention may incorporate any mechanical, chemical, and/or electrical technique for performing hemostasis.

As best shown in FIG. 1, the endoscope 20 includes an elongated tubular body 24 having a proximal end (not shown) adapted to be coupled to a conventional control and display system (not shown), a distal tip 36 provided at the endoscope's distal end, and a central lumen (not shown) disposed therebetween. The distal tip 36 is shown as a generally cylindrical member, and houses the vision system of the endoscope 20. The vision system includes LED's or another illumination source, such as fiber optic channels, lens, and CMOS or CDD image sensor conventionally arranged as known in the art. The illumination source and the image sensor are disposed in the imaging port 54 and the illumination port 58, respectively. The distal tip 36 further includes a insufflation/irrigation port 64 fluidly communicating with a supply lumen for supplying air/gas/liquid to regions positioned at the distal end of the endoscope 20.

The distal tip 36 further includes a hemostasis device 40 carried by or otherwise associated therewith. In the embodiment shown in FIG. 1, the hemostasis device 40 is configured as an electrode probe 42 projecting from the distal end face 46 of the distal tip 36. As best shown in FIG. 1, the electrode probe 42 is a monopolar probe. The monopolar electrode probe 42 includes a cylindrical body portion 66 having a hemispherical distal end tip 70. A discrete spiral electrode 74 is disposed on the outer surface of the body portion 66 and the end tip 70 and connects to an electrical lead (not shown) that supplies RF energy to the electrode 74 from a radio frequency (RF) energy generator housed exterior the endoscope 20. The monopolar electrode 74 is used in conjunction with a second electrode (not shown) connected to an exterior portion of the body, as known in the art. In use, the electrode probe 42 is placed on or in proximity to the site of internal bleeding and RF energy is supplied thereto for heating the area surrounding the site of internal bleeding, as known in the art.

Although the electrode probe 42 is described as monopolar, it is well understood in the art that the electrode probe 42 can be configured as a bipolar electrode probe with the addition of a second discrete electrode (not shown), such as a spiral electrode. Alternatively, the electrode probe 42 may be connected to a source of ultrasound energy for performing the desired hemostasis.

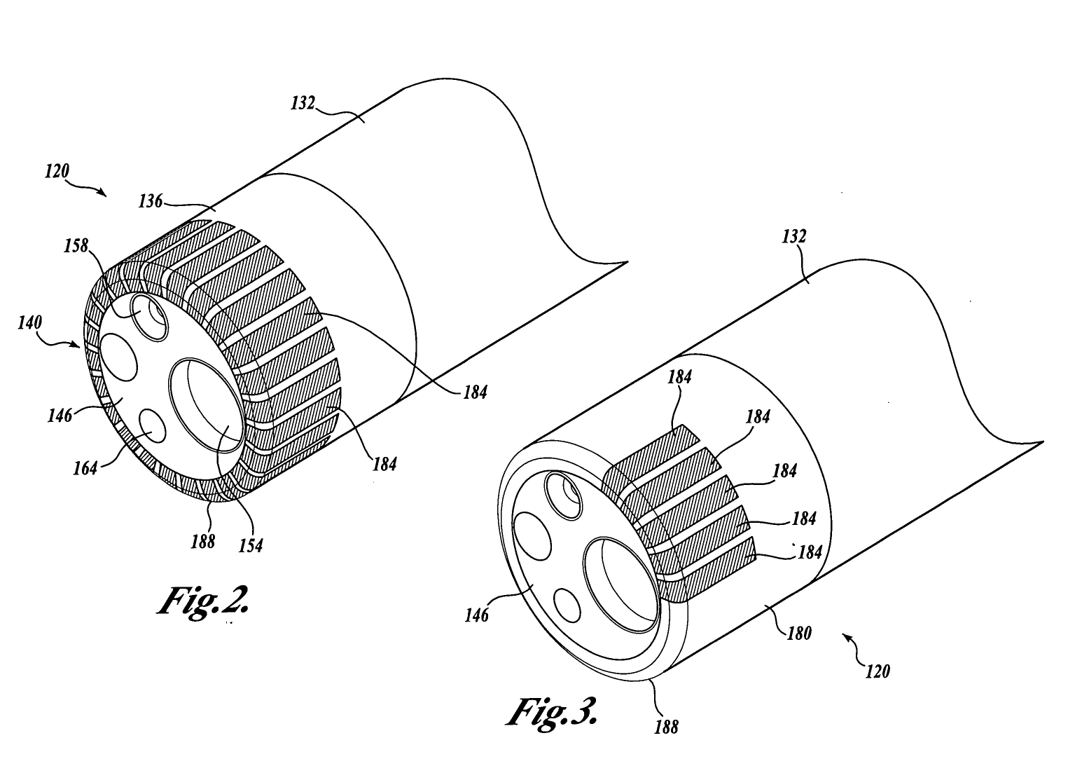

FIG. 2 illustrates an alternative embodiment of an endoscope 120 formed in accordance with aspects of the present invention. The endoscope 120 is substantially similar in materials, construction, and operation as endoscope 20, except for the differences that will now be described. As best shown in FIG. 2, the electrode probe is omitted, and in its stead the distal end face 146 of the distal tip 136 and/or the distal side surface 180 of the distal tip 136 may include either bipolar or monopolar electrodes 184 for supplying RF energy to an interior of the patient. In the embodiment shown, a plurality of electrodes 184 are disposed around the peripheral edge 188 of the distal tip 136 and along a portion of the distal side surface 180 of the distal tip 136. While the electrodes 184 are shown extending around the entire peripheral edge 188 of the distal tip 136, it will be appreciated that the electrodes may be disposed along any portion or portions of the peripheral edge 188 and/or side surface 180, as best shown in FIG. 3.

The electrodes 184 may be flush mounted on the endoscope 120 or may be raised slightly from the outer surface thereof. The electrodes 184 are electrically isolated from one another. In one embodiment, the electrodes 184 may be electrically isolated by a dielectric material, such as mica or plastic, disposed therebetween. Alternatively, the distal tip 136 could be made of a di-electric material, portions of which separate the electrodes 184. Each electrode 184 is electrically connected to an RF energy generator disposed external the endoscope 120. It will be appreciated that the electrodes may be connected to the RF energy generator in a bipolar configuration, or may be connected to the RF energy generator in a monopolar configuration and used in conjunction with a second electrode (not shown) connected to an exterior portion of the body, as known in the art.

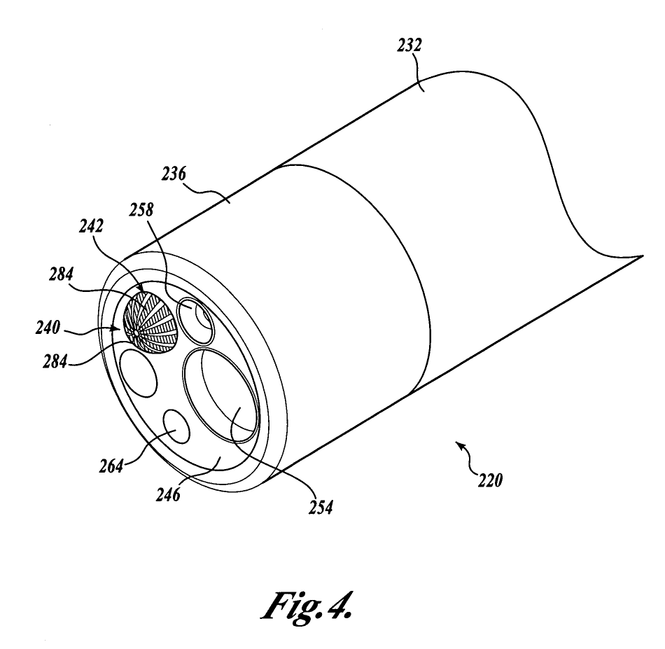

FIG. 4 illustrates another alternative embodiments of an endoscope 220 formed in accordance with aspects of the present invention. The endoscope 220 is substantially similar in materials, construction, and operation as endoscopes 20 and 120, except for the differences that will now be described. As best shown in FIG. 4, the hemostasis device 240 is a domed shaped electrode assembly 242 comprised of a plurality of electrodes 284. The electrode assembly 242 is disposed at the distal end face 246 of the distal tip 236. Each electrode 284 is electrically isolated from adjacent electrodes. In one embodiment, dielectric spacers are positioned in-between adjacent electrodes 284. Each electrode 284 is electrically connected to an RF energy generator disposed external the endoscope 220. It will be appreciated that the electrodes may be connected to the RF energy generator in a bipolar configuration, or may be connected to the RF energy generator in a monopolar configuration and used in conjunction with a second electrode (not shown) connected to an exterior portion of the body, as known in the art.

In the exemplary embodiments shown in FIGS. 1-4, the hemostasis devices are formed as part of or fixedly coupled to the distal tips of the endoscopes. However, other configurations of an endoscope having an associated hemostasis device are possible, as will now be described in detail. Referring now to FIG. 5, there is shown a partial cross-sectional view of another embodiment of an endoscope 320 formed in accordance with aspects of the present invention. As best shown in FIG. 5, the distal tip 336 includes a projecting member 358 that defines a cavity 360 from which an electrode probe 342 may be selectively advanced. While a projecting member is shown, it will be appreciated that the electrode probe 342 may be housed in a cavity formed in a conventionally shaped distal tip.

The electrode probe 342 includes a cylindrical body portion 366 having a hemispherical distal end tip 370. A discrete spiral electrode 374 is disposed on the outer surface of the body portion 366 and the end tip 370. The electrode probe 342 is dimensioned so as to slidably fit within the cavity 360 when retracted. The proximal end of the probe 342 is functionally connected to an advancer 382, such as a push-pull stylet, that retracts and advances the electrode probe 342 into and out of the cavity 360. The advancer 382 is constructed to exert force in both tension and compression. The advancer 382 is preferably formed of an electrical conductor material so that the advancer 382 may also function as the electrical lead connecting the electrode probe 342 to a source of RF energy. Alternatively, the advancer 382 may include a discrete electrical transmission structure for connecting the electrodes of the probe 342 to a RF energy generator.

While embodiments of the present invention were shown and described as utilizing an RF electrode probe or electrode arrays as the hemostasis device, other hemostasis devices using mechanical, chemical, or electrical modalities may be practiced with and are within the scope of the present invention. Several examples of mechanical modalities include, but are not limited to, clips, sutures, patches, and staples. With regard to chemical modalities, the endoscope may be configured to discharge a blood clotting agent or hemostat, such as alcohol or fibrinogen, from a discharge port located at the distal end face of the distal tip, such as the irrigation/insufflation port. Alternatively, the distal tip of the endoscope could be configured with a swellable hydrogel coating that could selectively release the hemostatic agents via compression against the passageway wall or with other trigger mechanisms, such as heat.

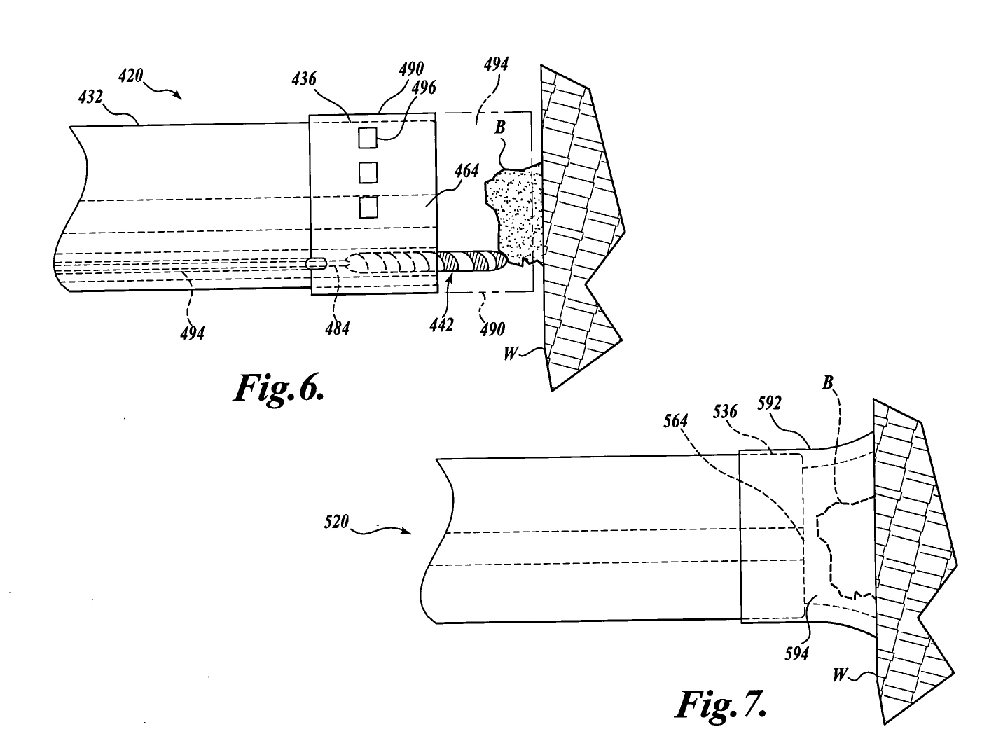

In some instances during endoscopy, a physician detects through the images obtained by an endoscope that a blood clot has formed at a site of previous internal bleeding (e.g. internal wound) due to the patient's normal physiological response. In such cases, it is preferable to treat the site and to perform subsequent hemostasis to ensure that the internal bleeding has stopped. Thus, in accordance with another aspect of the present invention, an endoscope may also be configured to treat such an internal site. Turning now to FIG. 6, there is shown a partial perspective view of the distal end of one exemplary embodiment of an endoscope 420 proximate the location of a blood clot B on internal passageway wall W. As will be described below, the endoscope 420 is configured for treating the site by: (1) cleaning the site; (2) removing the blood clot; and (3) performing hemostasis.

The endoscope 420 is substantially similar in materials, construction, and operation as endoscope 320, except for the differences that will now be described. As best shown in FIG. 6, the endoscope 420 further includes an outer peripheral collar 490 concentrically arranged with the distal tip 436. The collar 490 is slidably connected to the distal end of the endoscope 420. The collar 490 is slidably movable in a selective manner from a retracted position shown in FIG. 6, wherein the collar 490 surrounds the distal tip 436, to an extended position shown in phantom in FIG. 6, wherein the collar 490 is advanced past the distal end face, thereby forming an open ended inner cavity 494.

Movement of the collar 490 may be effected by an advancer 494, such as a push-pull stylet, that extends through the endoscope 420 and connects to the collar 490 at its proximal end. As the collar 490 advances, the collar 490 slidably seats over the distal tip 436, thereby forming a somewhat fluid tight inner cavity 494. The collar 490 further includes one way flap valves 496 or other one way valves around its perimeter to allow fluid and debris to escape from within the inner cavity 494 of the extended collar 490, but will prohibit fluids and debris from entering the inner cavity 494 of the extended collar 490.

The endoscope 420 further includes an extendible electrode probe 442 similar in construction and operation as the probe 342 in FIG. 5 that advances from a cavity formed in the distal tip 436. The advancer structure 482, such as a stylet, is slidably disposed in concentric relationship within advancer 494.

In use, when the physician spots a blood clot that needs to be treated as the endoscope 420 is routed through a passageway of the patient's body, the endoscope 420 is maneuvered into position by conventional steering wires/steering mechanism. Once in position, the collar 490 is advanced to the extended position via the advancer 494, whereby the collar 490 covers the existing clot B and preferably forms a seal between the passageway wall W and the end of the collar 490. Next, high pressure fluid may be selectively discharged from the irrigation/insufflation port 464. Alternatively, if space allows, a separate high pressure discharge nozzle may be positioned at the distal tip of the endoscope and supplied with a source of high pressure fluid exterior the endoscope. In either case, the high pressure jet of fluid is directed at the existing clot B for removal thereof. As the fluid jet is discharged from the distal end of the endoscope 420 for removing the clot, the clot material, other debris, and the fluid may exit the interior cavity of the collar through the one-way valves 296. After the clot B is removed, the collar 490 may be retracted, and the site of previous bleeding may be treated by the electrode probe 442.

It will be appreciated that the site of the blood clot B may be cleaned prior to removing the clot. In this case, a cleaning fluid, such as saline, may be discharged from the irrigation/insufflation port 464 or other port provided by the endoscope 420. It will be appreciated that appropriate plumbing, controllable valves, and pumps may be arranged in a conventional manner for providing the irrigation port the ability to selectively discharge irrigation fluid, air, and cleaning fluid. It will also be appreciated that the irrigation port/insufflation port may discharge a chemical agent, such as a thrombolytic agent, for blood clot removal. As is known in the art, such thrombolytic agents dissolve blood clots. Some examples of thrombolytic agents are tissue plasminogen activator (TPA) and streptokinase. Alternatively, ultrasound energy may be used to remove the blood clot. It will further be appreciated that embodiments of the endoscope 420 may use other hemostasis modalities than the electrode probe to ensure the stoppage of bleeding, such as chemical agents, clips, staples, sutures, etc.

FIG. 7 illustrates a partial perspective view of another embodiment of an endoscope 520 formed in accordance with the present invention. The endoscope 520 is substantially similar in materials, construction, and operation as endoscope 20, 320 and 420, except for the differences that will now be described. As best shown in FIG. 7, the endoscope 520 further includes a flexible collar 590 that extends from the end of the distal tip 536 of the endoscope 520. The flexible collar 590 is generally sheath-like, defining an interior, open ended cavity 594. The endoscope 520 further includes a hemostasis device, however, for ease of illustration, the hemostasis device, such as the extendible electrode probe shown in FIG. 7, has not been shown. Alternatively, as was described above, the discharge port 564 may be used to clean, remove, and/or stop internal bleeding.

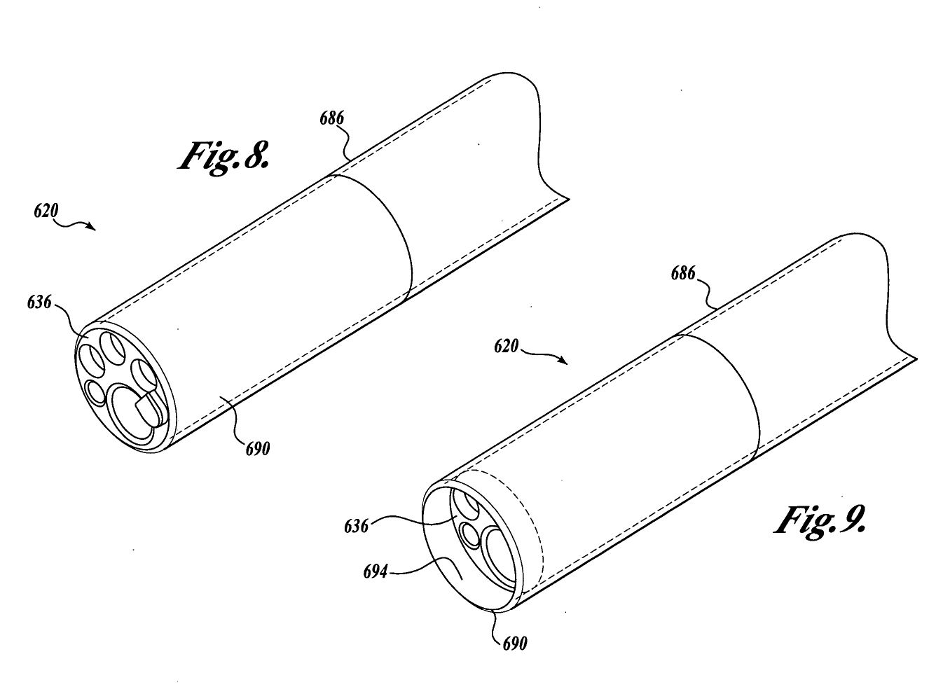

FIGS. 8 and 9 illustrate a partial perspective view of another embodiment of an endoscope 620 formed in accordance with the present invention. The endoscope 620 is substantially similar in materials, construction, and operation as endoscope 20, 320, 420, and 520 except for the differences that will now be described. As best shown in FIG. 8, the endoscope includes a distal shaft portion 686, a flexible collar 690, and a distal tip 636. The distal tip 636 is slidably disposed with respect to the flexible collar 690. In use, the distal tip 636 is slidably movable in a selective manner from an extended position shown in FIG. 8, wherein the collar 690 surrounds the distal tip 636 and is substantially flush therewith, to a retracted position shown in FIG. 9, wherein the distal tip 636 is withdrawn into the collar 690, thereby forming an open ended inner cavity 694.

While the preferred embodiment of the invention has been illustrated and described, it will be appreciated that various changes can be made therein without departing from the spirit and scope of the invention. For example, the electrode probe or other portions of the distal tip may be configured to delivery therapeutic drugs as well as blood clotting drugs. It is therefore intended that the scope of the invention be determined from the following claims and equivalents thereof.

The embodiments of the invention in which an exclusive property or privilege is claimed are defined as follows:

1. An endoscope, comprising:
an elongated shaft body having a proximal end and a distal end;
a distal tip section coupled to the distal end of the body; and
a hemostasis device carried on the body and positioned proximal the distal tip section.

2. The endoscope of claim 1, further comprising an imaging system associated with the distal tip section.

3. The endoscope of claim 1, wherein the hemostasis device is fixedly coupled at the distal end of the shaft body.

4. The endoscope of claim 1, wherein the hemostasis device projects from the distal end of the shaft body.

5. The endoscope of claim 1, wherein the hemostasis device is movably connected to the shaft body, and extendible past the distal end of the shaft body.

6. The endoscope of claim 1, wherein the hemostasis device is an electrode probe.

7. The endoscope of claim 6, wherein the electrode probe is bipolar or monopolar.

8. The endoscope of claim 1, wherein the hemostasis device comprises at least one electrode adapted to be connected to a source of RF or ultrasound energy.

9. The endoscope of claim 8, wherein the distal tip section defines a side surface and a distal end face, the electrode being disposed on the side surface and/or the distal end face the distal tip section.

10. The endoscope of claim 1, wherein the hemostasis device is a domed shaped electrode assembly.

11. The endoscope of claim 1, wherein the hemostasis device performs hemostasis using electrical, mechanical, or chemical modalities.

12. The endoscope of claim 1, wherein the hemostasis device is selected from the group consisting of staples, sutures, clips, patches, blood clotting agents, RF probes, and ultrasound energy devices.

13. An endoscope, comprising:
an elongated, flexible body having a proximal end and a distal end;
a distal tip coupled to the distal end of the body; and
hemostasis means carried on the body and positioned proximal the distal tip.

14. The endoscope of claim 13, wherein the hemostasis means is selected from the group consisting of staples, sutures, clips, patches, blood clotting agents, RF probes, and ultrasound energy devices.

15. A method of treatment using an endoscope with hemostasis capabilities, comprising:
routing an endoscope having an associated hemostasis device through a passageway to an internal wound site; and
performing hemostasis at the internal wound site with the hemostasis device.

16. The method of claim 15, further including locating a blood clot within the passageway with the endoscope.

17. The method of claim 16, further including removing the blood clot.

18. The method claim 17, wherein removing the blood clot includes mechanically removing the blood clot.

19. The method of claim 17, wherein removing the blood clot includes chemically removing the blood clot.

20. The method of claim 17, wherein removing the blood clot includes contacting the blood clot with a pressurized fluid stream.

21. The method of claim 17, wherein removing the blood clot includes contacting the blood clot with a fluid stream comprising a thrombolytic agent.

22. The method of claim 17, further comprising
covering a least a portion of a blood clot positioned on the wall of the passageway prior to removal of the blood clot.

23. The method of claim 22, wherein covering the blood clot includes advancing a collar disposed over the distal end of the endoscope.

24. The method of claim 22, wherein covering the blood clot includes positioning the endoscope in relation to the blood clot so that a flexible sheath associated with the distal tip of the endoscope is placed over the blood clot.

25. The method of claim 17, further including cleaning the area proximate the blood clot prior to blood clot removal.

26. The method of claim 15, wherein the hemostasis device is selected from the group consisting of staples, sutures, clips, patches, blood clotting agents, RF probes, RF electrodes, and ultrasound energy devices.
